(19) United States
(12) Patent Application Publication
(10) Pub. No.: US 2012/0065512 A1
(43) Pub. Date: Mar. 15, 2012

(54) ULTRASONIC DIAGNOSTIC APPARATUS AND ULTRASONIC IMAGE PROCESSNG APPARATUS

(76) Inventors: Kenji HAMADA, Otawara-shi (JP); Cong YAO, Otawara-shi (JP)

(21) Appl. No.: 13/230,201

(22) Filed: Sep. 12, 2011

(30) Foreign Application Priority Data
Sep. 13, 2010 (JP) 2010-204774

(51) Int. Cl. A61B 8/13

(52) U.S. Cl. 600/443

(57) ABSTRACT

According to one embodiment, there is provided an ultrasonic diagnostic apparatus Which comprises a volume data acquisition unit configured to acquire volume data by scanning a three-dimensional region including at least part of a fetus with an ultrasonic wave, a detection unit configured to detect NT data which corresponds to an NT region of the fetus and a longitudinal direction of the NT region with reference to an image which is generated by using the volume data and corresponds to a predetermined sagittal slice including the NT region, a measurement unit configured to measure thicknesses with respect to positions in the NT region and a line-of-sight direction with reference to the longitudinal direction and an image generation unit configured to generate an image indicating at least one of thicknesses of the NT region.

### CROSS-REFERENCE TO RELATED APPLICATIONS

This application is based upon and claims the benefit of priority from prior Japanese Patent Application No. 2010-204774, filed Sep. 13, 2010, the entire contents of which are incorporated herein by reference.

### FIELD

Embodiments described herein relate generally to an ultrasonic diagnostic apparatus and an ultrasonic image processing apparatus.

### BACKGROUND

Embodiments are directed to an ultrasonic diagnostic apparatus and the like which visualize and diagnose the interior of a living body by using ultrasonic waves and, more specifically, to an ultrasonic diagnostic apparatus and an ultrasonic image processing apparatus which perform NT (Nuchal Translucency: a region existing in the posterior region of the neck which is a target when, for example, a fetus in the early stage of pregnancy is to be ultrasonically diagnosed) measurement for an acquired image.

Ultrasonic diagnosis is performed by observing the pulsation of the heart, a slice of an organ, or the movement of a fetus in real time by bringing an ultrasonic probe into contact with the surface of the body. This system is smaller in size than other diagnostic apparatuses such as X-ray, CT, and MRI apparatuses, and uses a simple technique which facilitates examination to be performed by moving the apparatus to the bed side. Furthermore, ultrasonic diagnosis is free from the influence of radiation exposure unlike diagnosis using X-rays and is highly safe, and hence allows repetitive examination. Such ultrasonic diagnostic apparatuses are used in obstetric treatment, fetal diagnosis, treatment at home, and the like.

For example, NT measurement using an ultrasonic diagnostic apparatus in fetal diagnosis is known as an effective unit for checking the possibility of a genetic disorder. This measurement refers to the measurement accuracy, which is 0.1 mm, the gestational age (GA) of a fetus, which ranges from 11 weeks to 13⁺⁶ weeks, the crown-rump length (CRL), which ranges from 45 mm to 84 mm, the body position of a fetus, image size, and the like. Training is required to perform accurate measurement.

### BRIEF DESCRIPTION OF THE DRAWINGS

FIG. 1 is a block diagram of an ultrasonic diagnostic apparatus 1 according to an embodiment;

FIG. 2 is a modification of the block diagram of the ultrasonic diagnostic apparatus 1 according to this embodiment;

FIG. 3 is a flowchart showing a procedure for NT measurement support processing according to this embodiment;

FIG. 4 is a view for explaining how a reference region and a reference point are set on a sagittal image;

FIG. 5 is a view for explaining an example of a line-of-sight direction decision processing;

FIG. 6 is a view for explaining another example of a line-of-sight direction decision processing;

FIG. 7 is a view for explaining an example of measurement processing for the thickness of an NT in the line-of-sight direction;

FIG. 8 is a view for explaining another example of measurement processing for the thickness of an NT in the line-of-sight direction;

FIG. 9 is a view for explaining generation processing of a three-dimensional image including an NT region;

FIG. 10 is a view for explaining generation processing of a three-dimensional image including an NT region;

FIG. 11 is a view for explaining line-of-sight direction/NT data orientation adjustment processing;

FIG. 12 is a view showing an example of an icon for angle adjustment which is used for line-of-sight direction/NT data orientation adjustment processing;

FIG. 13A is a view showing an example of a display form of the maximum value of an NT and a three-dimensional image;

FIG. 13B and FIG. 13C are views each of which shows example of another display form of the maximum value of an NT and a three-dimensional image;

FIG. 14 is a view showing an example of a measurement region set in a three-dimensional image including an NT region;

FIG. 15 is a view showing another example of the display form of the maximum value of the NT and the three-dimensional image; and

FIG. 16 is a view showing another example of a measurement region set in a three-dimensional image including an NT region.

### DETAILED DESCRIPTION

In general, according to one embodiment, there is provided an ultrasonic diagnostic apparatus which comprises: a volume data acquisition unit configured to acquire volume data by scanning a three-dimensional region including at least part of a fetus with an ultrasonic wave; a detection unit configured to detect NT data, of the volume data, which corresponds to an NT region of the fetus, and configured to detect a longitudinal direction of the NT region with reference to an image which is generated by using the volume data and corresponds to a predetermined sagittal slice including the NT region; a measurement unit configured to measure a plurality of thicknesses with respect to a plurality of positions in the NT region by using the NT data and a line-of-sight direction with reference to the longitudinal direction; an image generation unit configured to generate an image indicating at least one of thicknesses of the NT region by using the NT data and the line-of-sight direction; and a display unit configured to display a thickness with respect to at least one of positions of the NT region and the image.

An embodiment will be described below with reference to the accompanying drawing. Note that the same reference numerals in the following description denote constituent elements having almost the same functions and arrangements, and a repetitive description will be made only when required.

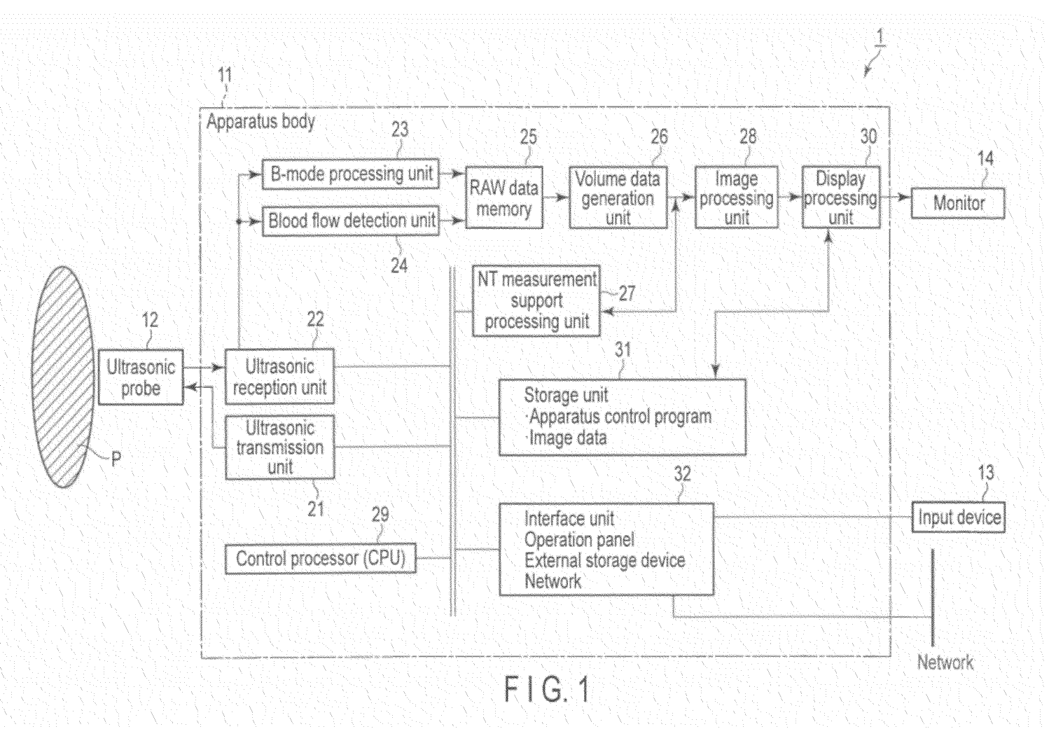

FIG. 1 is a block diagram showing the arrangement of an ultrasonic diagnostic apparatus 1 according to this embodiment. As shown in FIG. 1, the ultrasonic diagnostic apparatus 1 includes an ultrasonic probe 12, an input device 13, a monitor 14, an ultrasonic transmission unit 21, an ultrasonic reception unit 22, a B-mode processing unit 23, a blood flow detection unit 24, a RAW data memory 25, a volume data generation unit 26, an NT measurement support processing unit 27, an image processing unit 28, a control processor (CPU) 29, a display processing unit 30, a storage unit 31, and an interface unit 32. The function of each constituent element will be described below.

The ultrasonic probe 12 is a device (probe) which transmits ultrasonic waves to an object and receives reflected waves from the object based on the transmitted ultrasonic waves. The ultrasonic probe 12 has, on its distal end, a plurality of arrays of piezoelectric transducers, a matching layer, a backing member, and the like. The piezoelectric transducers of the ultrasonic probe 12 transmit ultrasonic waves in a predetermined direction in a scan region based on driving signals from the ultrasonic transmission unit 21, and convert reflected waves from the object into electrical signals. The matching layer is an intermediate layer provided for the piezoelectric transducers to make ultrasonic energy efficiently propagate. The backing member prevents ultrasonic waves from propagating backward from the piezoelectric transducers. When the ultrasonic probe 12 transmits an ultrasonic wave to an object, the transmitted ultrasonic wave is sequentially reflected by a discontinuity surface of acoustic impedance of internal body tissue, and is received as an echo signal by the ultrasonic probe 12. The amplitude of this echo signal depends on an acoustic impedance difference on the discontinuity surface by which the echo signal is reflected. The echo produced when a transmitted ultrasonic pulse is reflected by the surface of a moving blood flow is subjected to a frequency shift depending on the velocity component of the moving body in the ultrasonic transmission direction due to the Doppler effect.

Note that the ultrasonic probe 12 according to this embodiment is a two-dimensional array probe (which includes a plurality of ultrasonic transducers arranged in a two-dimensional matrix) or a mechanical 4D probe (which can execute ultrasonic scanning while mechanically swings an ultrasonic transducer array in a direction perpendicular to its array direction) as a device which can acquire volume data. This embodiment is not, however, limited to this, and may use, for example, a one-dimensional array probe as the ultrasonic probe 12. Using this probe can also acquire volume data by performing ultrasonic scanning while manually swinging the probe.

The input device 13 is connected to an apparatus body 11 and includes various types of switches, buttons, a trackball, a mouse, and a keyboard which are used to input, to the apparatus body 11, various types of instructions, conditions, an instruction to set a region of interest (ROI), various types of image quality condition setting instructions, and the like from an operator. When, for example, the operator operates the end button or FREEZE button of the input device 13, the transmission/reception of ultrasonic waves is terminated, and the ultrasonic diagnostic apparatus is set in a pause state.

The monitor 14 displays morphological information and blood flow information in the living body as images based on video signals from the image processing unit 28.

The ultrasonic transmission unit 21 includes a trigger generation circuit, delay circuit, and pulser circuit (none of which are shown). The trigger generation circuit repetitively generates trigger pulses for the formation of transmission ultrasonic waves at a predetermined rate frequency fr Hz (period: 1/fr sec). The delay circuit gives each trigger pulse a delay time necessary to focus an ultrasonic wave into a beam and determine transmission directivity for each channel. The pulser circuit applies a driving pulse to the probe 12 at the timing based on this trigger pulse.

The ultrasonic transmission unit 21 has a function of instantly changing a transmission frequency, transmission driving voltage, or the like to execute a predetermined scan sequence in accordance with an instruction from the control processor 29. In particular, the function of changing a transmission driving voltage is implemented by linear amplifier type transmission circuit capable of instantly switching its value or a mechanism of electrically switching a plurality of power supply units.

The ultrasonic reception unit 22 includes an amplifier circuit, A/D converter, and adder (none of which are shown). The amplifier circuit amplifies an echo signal received via the probe 12 for each channel. The A/D converter gives the amplified echo signals delay times necessary to determine reception directivities. The adder then performs addition processing for the signals. With this addition, a reflection component from a direction corresponding to the reception directivity of the echo signal is enhanced to form a composite beam for ultrasonic transmission/reception in accordance with reception directivity and transmission directivity.

The B-mode processing unit 23 receives an echo signal from the reception unit 22, and performs logarithmic amplification, envelope detection processing, and the like for the signal to generate data whose signal intensity is expressed by a luminance level.

The blood flow detection unit 24 detects a blood flow signal from the echo signal received from the ultrasonic reception unit 22 and generates blood flow data. The blood flow detection unit 24 generally detects a blood flow signal by CFM (Color Flow Mapping). In this case, the blood flow detection unit 24 analyzes the blood flow signal to obtain blood flow information such as an average velocity, variance, and power at multiple points as blood flow data.

The RAW data memory 25 generates B-mode RAW data as B-mode data on three-dimensional ultrasonic scanning lines by using a plurality of B-mode data received from the B-mode processing unit 23. The RAW data memory 25 also generates blood flow RAW data as blood flow data on three-dimensional ultrasonic scanning lines by using a plurality of blood flow data received from the blood flow detection unit 24. Note that in order to reduce noise and smoothly connect images, a three-dimensional filter may be inserted after the RAW data memory 25 to perform spatial smoothing.

The volume data generation unit 26 generates B-mode volume data from the B-mode RAW data received from the RAW data memory 25 by executing RAW-voxel conversion. The volume data generation unit 26 performs this RAW-voxel conversion by interpolation processing in consideration of spatial positional information to generate B-mode voxel data. Likewise, the volume data generation unit 26 generates blood flow volume data from blood flow RAW data received from the RAW data memory 25 by executing RAW-voxel conversion.

The NT measurement support processing unit 27 executes processing based on the NT measurement support function (to be described later) for the volume data generated by the volume data generation unit 26 under the control of the control processor 29.

The image processing unit 28 performs predetermined kinds of image processing such as volume rendering, multi planar reconstruction (MPR), and maximum intensity projection (MIP) for the volume data received from the volume data generation unit 26 and the NT measurement support processing unit 27. Note that in order to reduce noise and smoothly connect images, a two-dimensional filter may be inserted after the image processing unit 28 to perform spatial smoothing.

The control processor 29 has a function as an information processing apparatus (computer), and controls the operation of the main body of this ultrasonic diagnostic apparatus. The control processor 29 reads out a dedicated program for executing NT measurement support function (to be described later) from the storage unit 31 and expands the program in the memory which the processor has, thereby executing computation, control, and the like for various kinds of processing.

The display processing unit 30 executes dynamic range control, brightness control, contrast control, γ curve correction, RGB conversion, and the like for various kinds of image data generated and processed by the image processing unit 28.

The storage unit 31 stores the dedicated program for executing the NT measurement support function (to be described later), diagnosis information (patient ID, findings by doctors, and the like), a diagnosis protocol, transmission/reception conditions, a program for implementing a speckle removal function, a body mark generation program, and other data. The storage unit 31 is also used to store images in the RAW data memory 25, as needed. It is possible to transfer data in the storage unit 31 to an external peripheral device via the interface unit 32.

The interface unit 32 is an interface associated with the input device 13, a network, and a new external storage device (not shown). The interface unit 32 can transfer, via a network, data such as ultrasonic images, analysis results, and the like obtained by this apparatus to another apparatus.

### (NT Measurement Support Function)

The NT measurement support function of the ultrasonic diagnostic apparatus 1 will be described next. This function supports accurate NT measurement using volume data acquired by the ultrasonic diagnostic apparatus.

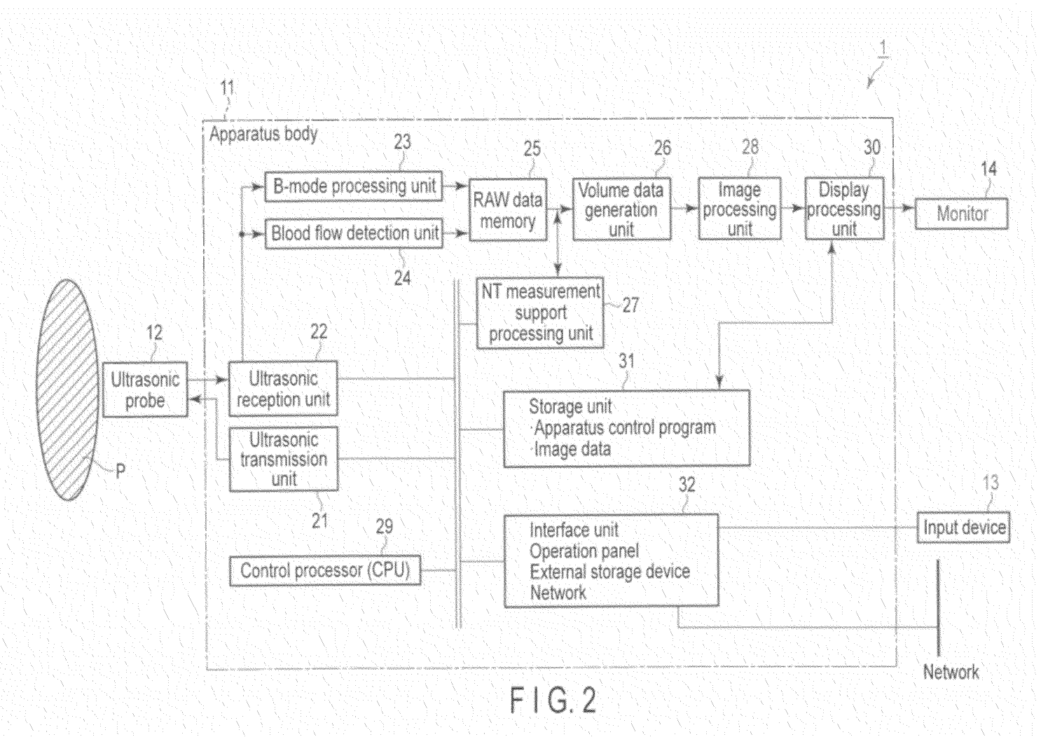

The following will exemplify a case in which processing (NT measurement support processing) based on the NT measurement support function is executed for the ultrasonic image generated by the volume data generation unit 26. However, this embodiment is not limited to this. For example, it is possible to execute NT measurement support processing for RAW data before it is input to the volume data generation unit 26. FIG. 2 shows an example of a block diagram of the ultrasonic diagnostic apparatus 1 in this case.

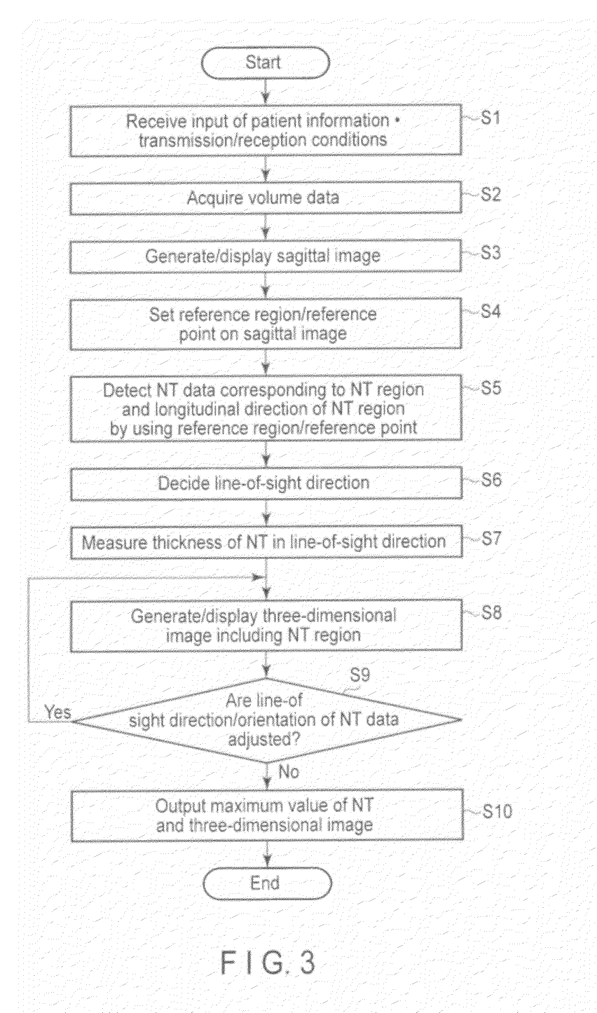

FIG. 3 is a flowchart showing a procedure for this NT measurement support function. The contents of processing in each step will be described below.

### [Reception of Input of Patient Information Transmission/Reception Conditions: Step S1]

The operator inputs patient information and selects transmission/reception conditions (a field angle for deciding the size of a region to be scanned, a focal position, a transmission voltage, and the like), an imaging mode for ultrasonic scanning on a predetermined region of an object, a scan sequence, and the like via the input device 13 (step S1). The storage unit 31 automatically stores various kinds of input and selected information and conditions and the like.

### [Acquisition of Volume Data: Step S2]

The operator brings the ultrasonic probe 12 into contact with a pregnant woman at a desired position, and executes ultrasonic scanning on a three-dimensional region including at least part of a fetus as a region to be scanned, thereby acquiring ultrasonic data. The acquired ultrasonic data are sequentially sent to the B-mode processing unit 23 via the ultrasonic reception unit 22. The B-mode processing unit 23 performs logarithmic amplification, envelope detection processing, and the like to generate image data whose signal intensity is expressed by a luminance for each frame. The RAW data memory 25 generates B-mode RAW data by using a plurality of B-mode data received from the B-mode processing unit 23. The volume data generation unit 26 generates B-mode volume data by executing RAW-voxel conversion for the B-mode RAW data received from the RAW data memory 25 (step S2).

### [Generation/Display of Sagittal Image: Step S3]

The image processing unit 28 generates a sagittal image of the fetus including an NT region (a region of the fetus which corresponds to an NT) by using the generated volume data. The monitor 14 displays the generated sagittal image in a predetermined form (step S3).

### [Setting of Reference Region/Reference Point on Sagittal Image: Step S4]

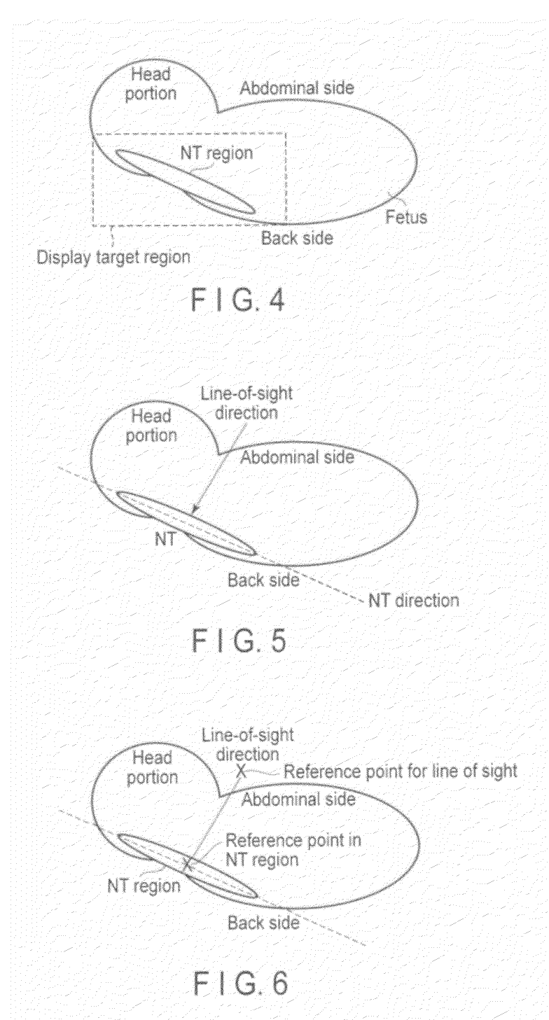

As shown in, for example, FIG. 4, when the operator selects a start button of NT measurement and inputs an NT region and a display target region on a sagittal image via the input device 13, the image processing unit 28 sets an NT region and a display target region on the sagittal image (step S4). However, the method of inputting/setting an NT region and a display region is not limited to this example. For example, when the operator designates an arbitrary point (e.g., a point near the center of an NT region) in an NT region on a sagittal image via the input device 13, the apparatus may automatically set an NT region with reference to this point. In addition, the apparatus may automatically set a display target region with reference to the set NT region.

Note that it is possible to change the positions, sizes, and directions of the NT region and display target region set in this step by input from the input device 13.

### [Detection of NT Data and Longitudinal Direction of NT Region: Step S5]

The NT measurement support processing unit 27 detects display target data corresponding to the set display target region and NT data (data corresponding to the NT region to be processed by the computer) corresponding to the NT region from the volume data. The NT measurement support processing unit 27 also detects the longitudinal direction (NT direction) of the NT region by using the detected NT data (step S5).

The method to be used to detect NT data corresponding to an NT region is not specifically limited. It is possible to use various methods, for example, a method of detecting NT data by threshold processing (segmentation) with a voxel value or a method of detecting the boundary of an NT region on each slice while shifting a sagittal slice within a display target region in the screen depth direction (the lateral direction of the fetus).

### [Decision of Line-of-Sight Direction: Step S6]

The NT measurement support processing unit 27 then decides a line-of-sight direction used for the measurement of a thickness in the NT direction and rendering as a normal direction to the NT direction (step S6). As shown in FIG. 5, assume that in this embodiment, an upper portion or right side of an image is regarded a viewpoint, and the line-of-sight direction from the abdominal side to the back side is used as a normal direction to the NT direction. However, it is possible to use either the direction from the back side to the abdominal side or the direction from the abdominal side to the back side as a line-of-sight direction, and hence to perform measurement regardless of the state (vertical orientation or horizontal orientation) in which the fetus is depicted. Alternatively, it is possible to designate, for example, a reference point for a line-of-sight direction at a desired position on a sagittal image and decide a line-of-sight direction from the reference point for the line-of-sight direction and the NT direction. In this case, as shown in FIG. 6, it is preferable to display a guideline indicating an NT direction and a designated reference point for a line-of-sight direction and to display a perpendicular drawn from the reference point for the line-of-sight direction to the guideline as a line-of-sight direction. It is also possible to decide, as a line-of-sight direction, a direction uniquely decided with reference to the NT direction instead of being limited to a normal direction to the NT direction.

### [Measurement of Thickness of NT in Line-of-Sight Direction: Step S7]

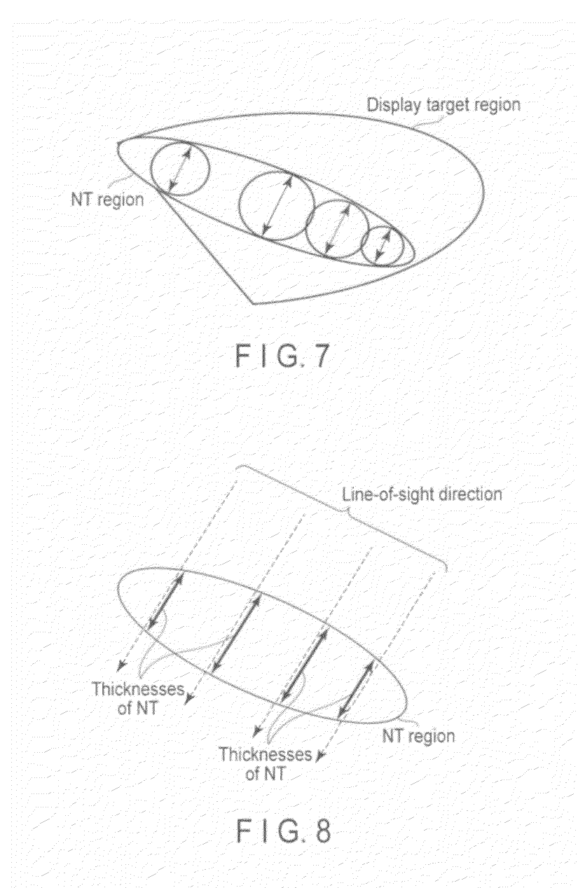

The NT measurement support processing unit 27 calculates the thickness of an NT in the line-of-sight direction by using the NT data and the line-of-sight direction (step S7). Note that the method to be used to calculate the thickness of an NT is not specifically limited. For example, as shown in FIG. 7, it is possible to set a plurality of spheres inscribed in an NT region and set the diameter of the largest sphere as the thickness of the NT. Alternatively, as shown in FIG. 8, it is possible to set a plurality of straight lines which are parallel to the line-of-sight direction and pass through an NT region and set the maximum value of the lengths of line segments cut by the NT region as the thickness of the NT in the line-of-sight direction.

### [Generation/Display of Three-Dimensional Image Including NT Region: Step S8]

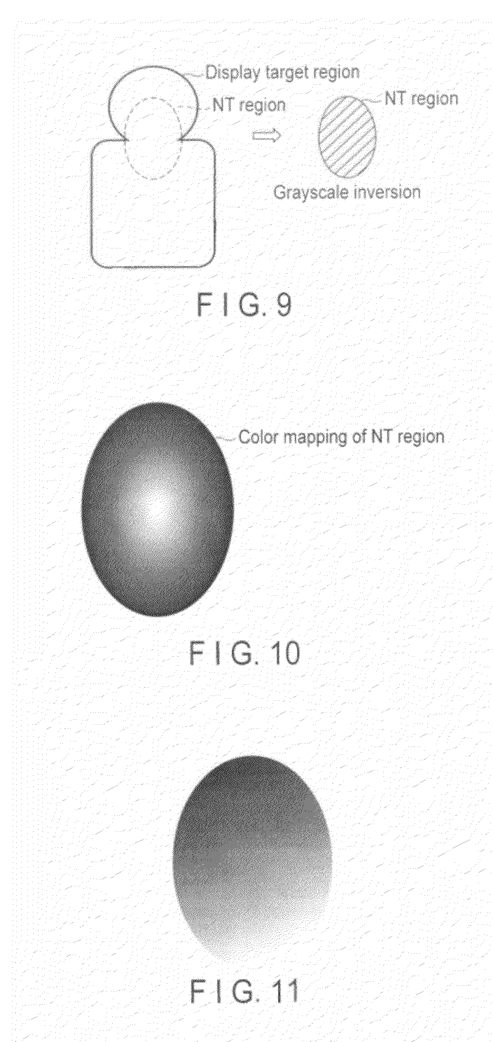

The image processing unit 28 then generates a cavity image or three-dimensional image including the NT region by executing rendering processing using the display target data. As shown in FIG. 9, the image processing unit 28 executes enhancement processing for making the NT region brighter than the remaining region by assigning a high value (white) to the voxels in the NT region while assigning a low value (black) to the remaining voxels or performing grayscale inversion processing or the like. As shown in FIG. 10, the image processing unit 28 also executes color mapping by assigning the NT region colors or densities (luminances) which vary depending on the thickness or variance value for each position. The monitor 14 displays the generated three-dimensional image in a predetermined form as a distribution image which shows a distribution of the thickness or variance value for each position (step S8).

### [Adjustment of Line-of-Sight Direction/Orientation of NT Data: Step S9]

If the inclination of the fetus in NT measurement is not correct, the generated NT region is displayed in an incomplete shape or the like as shown in, for example, FIG. 11. In such a case, it is possible to display the NT region in a complete shape or the like by adjusting at least one of the line-of-sight direction, the orientation of the NT data, and the position and orientation of a sagittal image.

That is, the image processing unit 28 changes at least one of the line-of-sight direction, the orientation of the NT data, and the position and orientation of the sagittal image, in response to an input from the input device 13, such that, for example, the position where the NT has the largest thickness coincides with the center of the display target region. In addition, the NT measurement support processing unit 27 and the image processing unit 28 execute steps S7 and S8 again by using the line-of-sight direction, NT data, and the like after the change. These processes are repeatedly executed until a desired three-dimensional image is acquired.

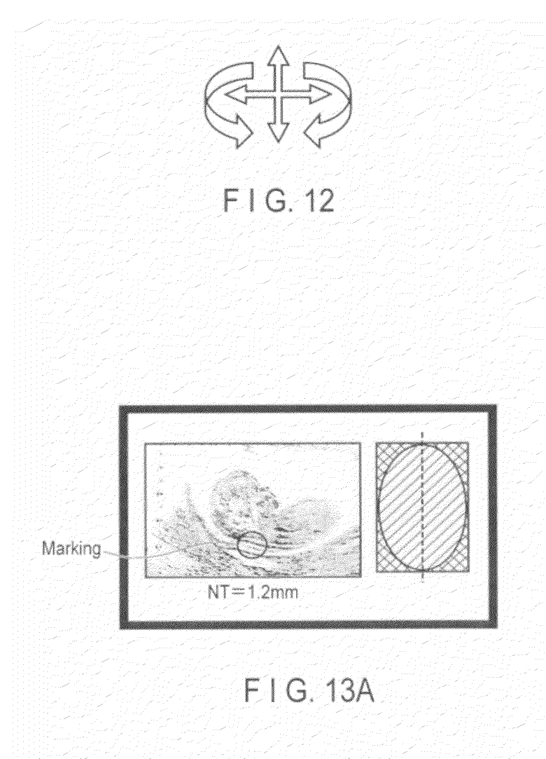

Note that it is preferable to adjust the orientation of NT data or sagittal image so as to avoid an excessive increase in changed angle (inclination) while visually checking a displayed sagittal image or three-dimensional image. Limiting a movable range in advance can prevent an excessive change. In addition, the apparatus can determine a case in which the position where an NT has the largest thickness does not coincide with the center of a display target region. In such a case, it is possible to actively prompt the operator to perform angle adjustment by displaying an icon for angle adjustment like that shown in FIG. 12 and explicitly indicating the angular direction to be adjusted in color.

### [Output of Maximum Value of NT and Three-Dimensional Image: Step S10]

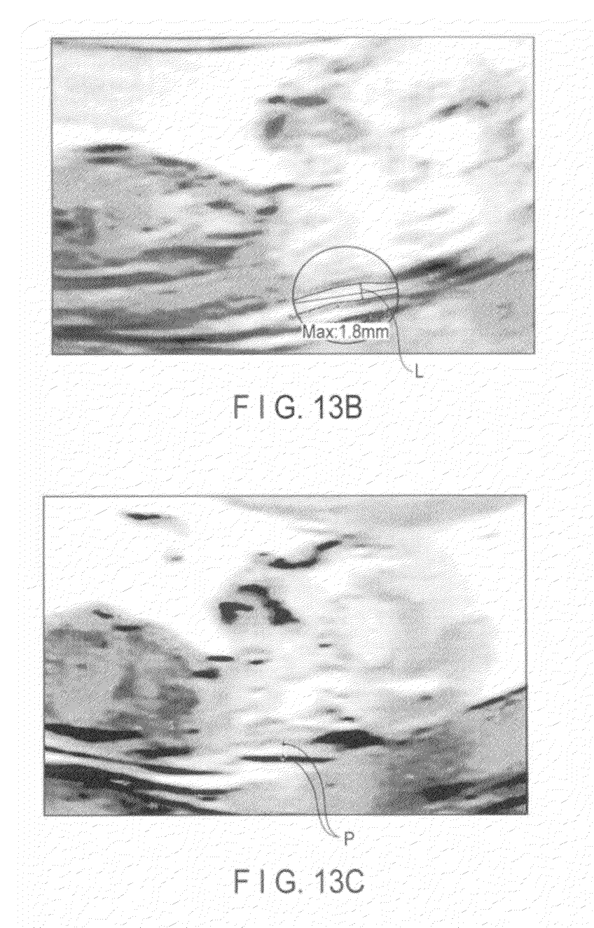

The generated three-dimensional image and the calculated NT thickness are output in a predetermined form and automatically stored in the storage unit 31 (step S10). The ultrasonic diagnostic apparatus according to this embodiment displays a sagittal image, a three-dimensional image including an NT region, and an NT thickness on the monitor 14 in, for example, the form shown in FIG. 13. Assume that if an NT region has concave and convex portions and varies in thickness, the apparatus displays the maxim value of the thicknesses (the circle on the sagittal image in FIG. 13A is a mark indicating a measurement position of the maximum value). Needless to say, the form in which the NT thickness is displayed is not limited to the example shown in FIG. 13A. For example, as shown in FIG. 13B, the NT thickness to be measured on the image may be displayed as line segment L, and a measurement range may be designated by use of the line segment. Furthermore, as shown in FIG. 13C, pointers P for defining one end and the other end of a measurement range may be displayed on an image, and an NT thickness may be displayed by use of pointers P. In this case, an obtained value is displayed in a predetermined form (in the example shown in FIG. 13C, the obtained value is displayed in the lower left portion of the screen).

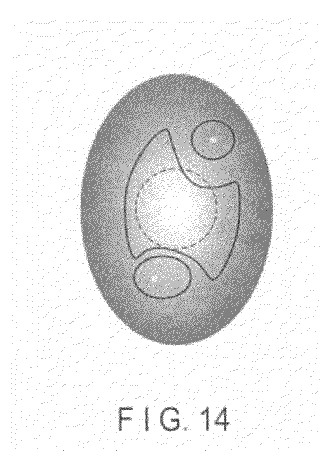

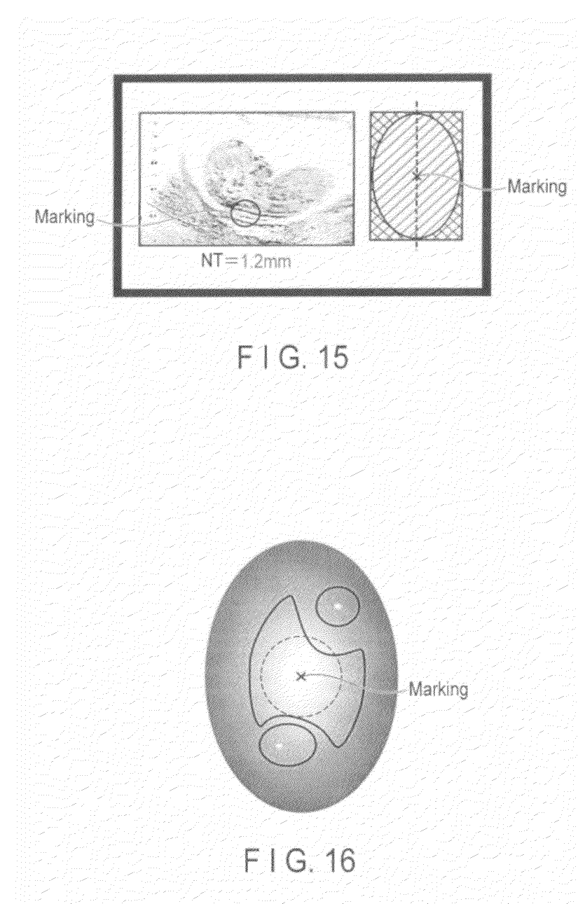

In addition, for example, as shown in FIG. 14, selecting a range as an NT measurement target on a displayed three-dimensional image can obtain a measurement value with higher accuracy. Furthermore, as shown in FIGS. 15 and 16, it is preferable to indicate a position corresponding to the maximum value in a three-dimensional image with a mark.

Note that if a GA input or measured in advance does not satisfy 11 weeks≦GA<14 weeks or a measured CRL does not satisfy 45 mm<CRL<84 mm, it is preferable to display a message indicating the corresponding information or add a predetermined mark indicating the corresponding information to the measured value.

### (Effects)

The ultrasonic diagnostic apparatus described above acquires volume data by ultrasonically scanning a three-dimensional region of a fetus which includes an NT region and sets a reference region or a reference point on a sagittal image obtained by using the volume data. The apparatus then detects NT data and an NT direction by using the set reference region or reference point, decides a line-of-sight direction by using the NT direction, and measures the maximum thickness of the NT region in the line-of-sight direction. It is therefore possible to measure the maximum thickness of the NT region more accurately than conventional measurement using a two-dimensional image.

This ultrasonic diagnostic apparatus also generates and displays a three-dimensional image having an NT region enhanced more than the remaining region by assigning a high value (white) to the voxels in the NT region while assigning a low value (black) to the remaining voxels or performing grayscale inversion processing or the like, or generates and displays a three-dimensional image by executing color mapping by, for example, assigning colors or densities (luminances) to the NT region which vary depending on the thickness or variance value for each position. This can provide a three-dimensional image with high visibility, and hence can contribute to an improvement in the quality of diagnosis in NT measurement.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
a volume data acquisition unit configured to acquire volume data by scanning a three-dimensional region including at least part of a fetus with an ultrasonic wave;
a detection unit configured to detect NT data, of the volume data, which corresponds to an NT region of the fetus, and configured to detect a longitudinal direction of the NT region with reference to an image which is generated by using the volume data and corresponds to a predetermined sagittal slice including the NT region;
a measurement unit configured to measure a plurality of thicknesses with respect to a plurality of positions in the NT region by using the NT data and a line-of-sight direction with reference to the longitudinal direction;
an image generation unit configured to generate an image indicating at least one of thicknesses of the NT region by using the NT data and the line-of-sight direction; and
a display unit configured to display a thickness with respect to at least one of positions of the NT region and the image.

2. The apparatus of claim 1, wherein the image comprises a distribution image indicating a distribution of thicknesses of the NT region.

3. The apparatus of claim 2, wherein the distribution image comprises an image whose pixel values are decided in accordance with the thicknesses of the NT region.

4. The apparatus of claim 1, wherein the image comprises one of a volume rendering image, a color map image to which colors are assigned in accordance with the thicknesses of the NT region, and a grayscale image to which luminances are assigned in accordance with the thicknesses of the NT region.

5. The apparatus of claim 1, wherein the display unit displays a maximum value of the plurality of thicknesses of the NT region.

6. The apparatus of claim 1, wherein the display unit displays the image on which a position corresponding to the maximum value is marked.

7. The apparatus of claim 1, further comprising a input unit configured to input a region including at least part of the NT region or a point existing in the NT region with respect to an image corresponding to the predetermined sagittal slice,
wherein the detection unit detects the NT region with reference to the input region or the input point.

8. The apparatus of claim 1, wherein the detection unit detects the NT region by detecting a boundary of the NT region on each slice while shifting the predetermined sagittal slice in a direction perpendicular to the slice.

9. The apparatus of claim 1, further comprising a changing unit configured to change an orientation of the fetus displayed on an image corresponding to the predetermined sagittal slice by changing at least one of a position and an angle of the predetermined sagittal image,
wherein the detection unit detects NT data, of the volume data, which corresponds to the NT region, and a longitudinal direction of the NT region, with reference to an image corresponding to the sagittal slice after the change.

10. The apparatus of claim 1, wherein the measurement unit decides the line-of-sight direction by using a point input by an operator and the normal direction.

11. The apparatus of claim 1, wherein the image generation unit changes at least one of the line-of-sight direction and the orientation of the NT data such that a maximum value of thicknesses of the NT region is located at or near a center of the NT region, and generates the image by using the line of sight or the NT data after the change.

12. The apparatus of claim 1, wherein the image generation unit generates the three-dimensional image by inverting grayscale to increase brightness of the NT region.

13. The apparatus of claim 1, wherein the image generation unit generates the image by setting the NT data as a voxel value higher than that of other data.

14. An ultrasonic image processing apparatus comprising:
a storage unit configured to store volume data acquired by scanning a three-dimensional region including at least part of a fetus with an ultrasonic wave;
a detection unit configured to detect NT data, of the volume data, which corresponds to an NT region of the fetus, and configured to detect a longitudinal direction of the NT region with reference to an image which is generated by using the volume data and corresponds to a predetermined sagittal slice including the NT region;
a measurement unit configured to measure a plurality of thicknesses with respect to a plurality of positions in the NT region by using the NT data and a line-of-sight direction with reference to the longitudinal direction;
an image generation unit configured to generate an image indicating at least one of thicknesses of the NT region by using the NT data and the line-of-sight direction; and
a display unit configured to display a thickness with respect to at least one of positions of the NT region and the image.

15. The apparatus of claim 14, wherein the image comprises a distribution image indicating a distribution of thicknesses of the NT region.

16. The apparatus of claim 15, wherein the distribution image comprises an image whose pixel values are decided in accordance with the thicknesses of the NT region.

17. The apparatus of claim 15, wherein the image comprises one of a volume rendering image, a color map image to which colors are assigned in accordance with the thicknesses of the NT region, and a grayscale image to which luminances are assigned in accordance with the thicknesses of the NT region.

18. The apparatus of claim 14, wherein the display unit displays a maximum value of the plurality of thicknesses of the NT region.

19. The apparatus of claim 14, wherein the display unit displays the image on which a position corresponding to the maximum value is marked.

20. The apparatus of claim 14, further comprising a input unit configured to input a region including at least part of the NT region or a point existing in the NT region with respect to an image corresponding to the predetermined sagittal slice,
wherein the detection unit detects the NT region with reference to the input region or the input point.

21. The apparatus of claim 14, wherein the detection unit detects the NT region by detecting a boundary of the NT region on each slice while shifting the predetermined sagittal slice in a direction perpendicular to the slice.

22. The apparatus of claim 14, further comprising a changing unit configured to change an orientation of the fetus displayed on an image corresponding to the predetermined sagittal slice by changing at least one of a position and an angle of the predetermined sagittal image,
wherein the detection unit detects NT data, of the volume data, which corresponds to the NT region, and a longitudinal direction of the NT region, with reference to an image corresponding to the sagittal slice after the change.

23. The apparatus of claim 14, wherein the measurement unit decides the line-of-sight direction by using a point input by an operator and the normal direction.

24. The apparatus of claim 14, wherein the image generation unit changes at least one of the line-of-sight direction and the orientation of the NT data such that a maximum value of thicknesses of the NT region is located at or near a center of the NT region, and generates the image by using the line of sight or the NT data after the change.

25. The apparatus of claim 14, wherein the image generation unit generates the three-dimensional image by inverting grayscale to increase brightness of the NT region.

26. The apparatus of claim 14, wherein the image generation unit generates the image by setting the NT data as a voxel value higher than that of other data.
